(19) United States
(12) Patent Application Publication
(10) Pub. No.: US 2021/0161877 A1
(43) Pub. Date: Jun. 3, 2021

(54) GASTROPROTECTED, HYDROPHOBIC FORMULATION OF AT LEAST ONE ACTIVE PRINCIPLE AND METHOD FOR OBTAINING SAME

(71) Applicants: Karim IOUALALEN, SAINT ORENS DE GAMEVILLE (FR); Rose-Anne RAYNAL, SAINT ORENS DE GAMEVILLE (FR)

(72) Inventors: Karim IOUALALEN, SAINT ORENS DE GAMEVILLE (FR); Rose-Anne RAYNAL, SAINT ORENS DE GAMEVILLE (FR)

(21) Appl. No.: 16/978,140

(22) PCT Filed: Mar. 7, 2019
(86) PCT No.: PCT/FR2019/050515

§ 371 Date: Feb. 22, 2021

(30) Foreign Application Priority Data
Mar. 8, 2018 (FR) 1870252

(51) Int. Cl. A61K 31/4439; A23L 33/12; A23P 10/22; A23K 20/158; A23K 20/137; A23K 20/174; A23K 10/18; A23L 33/14; A23L 33/15; A23K 40/10; A61K 9/16; A61K 36/064; A61K 35/741; A61K 33/44; A61K 33/34; A61K 33/38; A61K 33/26; A61K 33/06; A61K 31/07; A61K 31/375; A61K 31/593; A61K 31/355; A61K 31/455; A61K 35/60

(52) U.S. Cl. CPC A61K 31/4439; A23V 2002/00; A23P 10/22; A23K 20/158; A23K 20/137; A23K 20/174; A23K 10/18; A23L 33/14; A23L 33/15; A23K 40/10; A61K 9/1617; A61K 9/1694; A61K 36/064; A61K 35/741; A61K 33/44; A61K 33/34; A61K 33/38; A61K 33/26; A61K 33/06; A61K 31/07; A61K 31/375; A61K 31/593; A61K 31/355; A61K 31/455; A61K 35/60; A23L 33/12

(57) ABSTRACT

The invention relates to a hydrophobic gastro-protected formulation of at least one active principle, characterized in that it is in the form of solid particles with a size of between 5 μm and 3500 μm, the solid particles of the formulation comprising:
at least one waxy excipient forming a waxy matrix; and
said at least one active principle chosen from the group formed from active principles for agrifood use, active principles for veterinary use, active principles for probiotic use and active principles for pharmaceutical use;
   the solid particles being strictly hydrophobic, non-hygroscopic, non-injectable, free of water, free of surfactants, free of emulsifiers, free of solvent, and free of shear-thinning polymer;
   the solid particles having a melting point of between 15° C. and 60° C.;
   said at least one active principle being uniformly distributed in the waxy matrix in a mass proportion of between 0.2% and 85%;
   said solid particles being suitable for protecting and stabilizing said at least one active principle in a gastric medium and being dispersible in an intestinal medium.

The invention relates to a novel formulation of active compounds or active principles in hydrophobic, divided, solid form for stabilizing and gastro-protecting while at the same time facilitating the administration of the formulated active principle. The invention also relates to a process for obtaining such a formulation. According to the invention, such a formulation is in the form of a divided solid, i.e. a powder. Such a formulation is hydrophobic, stabilized and gastro-protected, i.e. protected with respect to a gastric environment. Such a formulation according to the invention may be used for producing agrifood, biotechnological, veterinary, technical and pharmaceutical products.

Many compounds are used in powder form. Natural compounds obtained in raw form and those transformed into powder by fragmentation and milling according to the conventional techniques described in Randolph and Larson, “Theory of Particulate Processes”, 2nd Edition, Academic Press, N Y, 1988 and in J. A. Dodds, C. Frances, P. Guigon, A. Thomas “Méthodologies pour la Modélisation du Broyage Fin, Colloque sur Science et Technologie des Poudres [Methodologies for modelling fine milling, colloquium on the science and technology of powders]”, Lyon, France, November 1994 may be distinguished.

Natural compounds obtained by solvent extraction and synthetic and semisynthetic products, prepared by chemical synthesis, are recovered by the conventional phase of precipitation from solvent, as described in “Handbook of Industrial Crystallization” 2nd Edition, Allan S. Myerson, BH Editions, 2002.

For products in aqueous phase, other techniques based on solid/liquid separation enable the production of the active compounds in powder form. This is the case for freeze-drying which is described in detail in the article S. C Tsinontides, “Freeze-drying—principles and practice for successful scale-up to manufacturing”, International Journal of Pharmaceutics, volume 280, Issues 1-2, August 2004 pages 1-16.

The atomization technique, also known as spray-drying, is also employed in solid/liquid separation, as described in D. Kumar D. et al, “Powder Preparation via Spray Dryer”, Ceram. Forum Int, 5 (1988) 141-44.

Throughout the text hereinbelow, the term “active agent”, “active compound” or “active principle” means any compound for obtaining a physicochemical, technical, biological, pharmacological, physiological, pharmaceutical, cosmetic, agrifood or biotechnological effect.

Many active compounds or active principles in the form of dry powder obtained via such processes have a certain number of drawbacks. Sensitive active compounds or active principles can be degraded due to the effect of chemical and/or physical and/or biological factors. Examples that may be mentioned include the following degradation reactions: hydrolysis on contact with water, acidic or alkaline degradation, oxidation, photodegradation, enzymatic degradation, instability of the crystalline form.

These phenomena may appear at the time of manufacture, during storage or during their subsequent use.

Several solutions have been developed for addressing these difficulties.

One solution relates to modification of the preparation conditions. It is possible to limit the degradation during preparation by change of solvent giving a protective effect. Thus, to protect ascorbic acid during milling, EP1688130A1 proposes to perform this step in an anhydrous oily phase. Other techniques are directed towards protecting the active agent subsequently during storage or use.

The technical solution most widely used consists in implementing a protective system by encapsulating the compound using polymers, as described in U.S. Pat. No. 4,434,009.

These techniques are also applied for pharmaceutical active agents that are sensitive to hydrolysis or to acidic stress. These encapsulation or coating techniques are well known to those skilled in the art. Physical coating processes, based on spraying of the coating solution in a turbomixer or in a fluidized bed, as described in WO 00/30617 and WO 02/092106, on the one hand, and physicochemical coating based on coacervation or phase separation, as described in U.S. Pat. No. 3,341,416, on the other hand, may be distinguished. These techniques for coating pharmaceutical active agents make it possible to obtain powders of stabilized and protected active agents, endowed with sustained-release properties described in U.S. Pat. No. 6,660,382 and with stabilization and resistance to the very acidic gastric medium, described in EP 1051174.

However, these techniques for preparing and/or treating powders have a certain number of drawbacks.

Thus, the technique of milling in an oily or organic protective solvent involves obtaining a non-dry oily form. The removal of the oily phase is difficult and involves the use of solvents or of removal techniques by drying or evaporation. All these techniques are expensive and long, and do not enable the solvent residues to be removed. Finally, the efficiency is too poor and does not afford long-term protection against oxidation or protection against acidic degradation, for example in a gastric medium.

The technique of modification by surface treatment of active agent crystals and/or particles has the major drawback of inducing a chemical modification of the active agent. For pharmaceutical, nutritional and biological applications, the active agent thus treated is no longer identical to the initial active agent. It can therefore no longer be used.

Finally, coating or encapsulation technologies have, themselves too, a certain number of drawbacks:
the kinetics of dissolution and dispersion of the active agent are considerably modified,
the coated particles have a size from a few hundred micrometres to a few millimetres and are perceptible during absorption. In this case, their breakdown may lead to a poor taste during administration,
it is difficult to render these technologies compatible with the preparation of stable liquid forms,
these processes are complex, involve numerous steps, are expensive and are incompatible with the preparation of lyophilized forms or any process using an aqueous solvent.

These technologies for preparing and/or treating powders are thus not entirely satisfactory.

The invention relates to a hydrophobic gastro-protected formulation of at least one active principle, characterized in that it is in the form of solid particles with a size of between 5 μm and 3500 μm, the solid particles of the formulation comprising:
at least one waxy excipient forming a waxy matrix; and
said at least one active principle chosen from the group formed from active principles for agrifood use, active principles for veterinary use, active principles for probiotic use and active principles for pharmaceutical use;
   the solid particles being strictly hydrophobic, non-hygroscopic, non-injectable, free of water, free of surfactants, free of emulsifiers, free of solvent, and free of shear-thinning polymer;
   the solid particles having a melting point of between 15° C. and 60° C.;
   said at least one active principle being uniformly distributed in the waxy matrix in a mass proportion of between 0.2% and 85%;
   said solid particles being suitable for protecting and stabilizing said at least one active principle in a gastric medium and being dispersible in an intestinal medium.

Throughout the text, the terms “mixture”, “mixture containing the active compound”, “mixture containing the active principle”, “matrix containing the active agent or the active principle” and “product containing the active agent or the active principle” denote the liquid or solid resulting from the mixing of the constituent waxy excipient(s) of the waxy matrix after melting in the reactor and of at least one active principle.

The inventors have discovered, entirely unexpectedly, that the formulation method according to the invention makes it possible to obtain a stable powder of particles in which the active principle(s) are protected against aqueous, acidic, basic, oxidative and biological attack without encountering the drawbacks mentioned previously. The active principle(s) thus formulated may be administered orally without gastric degradation. This formulation method enables, for example, the production of foods containing gastro-protected probiotic compounds, i.e. compounds that are protected in a gastric environment, which are particularly advantageous for animal feed.

The formulation according to the invention formed from a waxy matrix powder containing at least one active principle is thus characterized in that it is:
composed of a waxy matrix protecting the active principle or active principle mixture,
strictly hydrophobic and gastro-protected,
free of aqueous or organic solvent. A formulation according to the invention is free of any trace of aqueous or organic solvent. It is possible, however, that a formulation according to the invention contains a trace, i.e. a residual amount at the detectability limit, of aqueous or organic solvent not resulting from an intentional addition of aqueous or organic solvent to the formulation. They may be aqueous or organic solvent residues introduced by the active principle(s).
free of surfactant or amphiphilic or detergent compounds,
free of polymers and of polymeric agent residues,
characterized by a uniform distribution of the active principle(s) in the waxy matrix of the solid particles, and with no radial gradient within the solid particles,
biocompatible and dispersible in intestinal medium,
the solid particles constituting the powder according to the invention cannot be administered parenterally.

Advantageously, the solid particles of the formulation are formed exclusively from at least one waxy excipient forming the waxy matrix, and from at least one active principle chosen from the group formed from active principles for agrifood use, active principles for veterinary use, active principles for probiotic use and active principles for pharmaceutical use.

The formulation formed from a powder according to the invention has increased stability under tropical storage conditions and/or in aqueous medium.

The formulation according to the invention is free of water and of any polymeric aqueous gel. In particular, the solid particles of the formulation according to the invention are free of residual peripheral polymeric compounds. They are free of any polymeric compound, notably of any aqueous polymeric compound, derived from a process for forming lipid particles. The formulation according to the invention is a dry formulation.

The solid particles of the formulation according to the invention have any form. In particular, they do not have a distribution of regular spherical form. They have a distribution of irregular forms and are not derived from a process for forming particles within an aqueous polymer matrix.

The waxy matrix of the solid particles is hydrophobic and consists of a mixture of water-insoluble hydrophobic compounds which are solid at room temperature and totally free of surfactant compounds, of solvent residues and of water which may be the cause of hydrolysis or oxidation reactions. One or more active principles which are compatible with the waxy excipient(s) used, in particular of lipid type, are added to this waxy matrix.

In certain embodiments, the formulation according to the invention is in the form of hydrophobic solid particles with a size of between 10 μm and 2500 μm (microns, micrometres), preferentially between 150 μm and 800 μm.

By way of example, at least one—notably each—waxy excipient may be chosen from the group formed of hydrophobic waxes or a mixture of hydrophobic waxes of plant, animal, synthetic and/or mineral origin but also of oils and hydrophobic compounds. At least one—notably each—waxy excipient is chosen to make it possible to adjust the melting point, the hardness, the physicochemical properties and the biological properties of the formulation, notably the biodegradability. The waxy matrix may contain, in addition to additives, one or more soluble or insoluble active principles such as mineral particles. According to the invention, use is made of mixtures of the waxy excipient(s) and of the active principle(s) with a melting point of between 15° C. and 60° C., notably between 25° C. and 60° C., preferentially between 32° C. and 52° C. According to the invention, use is made of one or more waxy excipients with a melting point of between 15° C. and 60° C., notably between 25° C. and 60° C., preferentially between 32° C. and 52° C. Advantageously, the solid particles have a melting point of between 25° C. and 60° C., preferentially between 32° C. and 52° C.

As waxy excipient, use may be made of at least one triglyceride, notably at least one triglyceride whose fatty acids contain from 8 to 30 carbon atoms (C8 to C30). It is also possible to use other waxy excipients such as fatty alcohols of high molecular weight, fatty acids (non-ionized and non-neutralized, in carboxylic acid form —COOH) which are preferentially linear and saturated C12 to C30 pairs, and esters of fatty acids and alcohols of high molecular weight, notably esters of C8 to C30 fatty acids and of C2 to C32 alcohols. In all cases, the waxy matrix and the mixture of the waxy matrix and of the active principle(s) are in solid form at room temperature or physiological temperature and characterized by the absence of water, by the absence of surfactant compounds, by hydrophobic behaviour, non-wettability with water and the absence of hygroscopicity. Throughout the text, the term “fatty acid(s)” denotes one or more non-ionized and non-neutralized fatty acids, i.e. in carboxylic acid form (—COOH).

In certain embodiments, at least one waxy excipient of the waxy matrix is chosen from the group formed from palm oil, carnauba waxes, candelilla waxes, esparto grass waxes, cocoa butter, plant waxes, beeswaxes and modified beeswaxes. In certain embodiments, the waxy matrix comprises—and notably consists exclusively—of waxes of natural origin. The fatty acids used according to the invention are in non-ionized and non-neutralized acid form (—COOH). Since the salts of these fatty acids form soaps, they cannot in any way be used since they would promote the wettability of the matrix.

In certain embodiments, at least one plant wax is chosen from the group formed from olive wax, rice wax, hydrogenated jojoba wax and absolute waxes of flowers.

In certain embodiments, at least one waxy excipient is chosen from the group formed from polyolefins, fatty acids (non-neutralized and non-ionized), esters of linear-chain fatty acids with a number of carbon atoms of between 4 and 30, for instance lauric acid, myristic acid, palmitic acid and stearic acid, esters of linear-chain fatty acids with a number of carbon atoms of between 4 and 30 and hydrophobic lipids.

As waxy excipient, use may be made of at least one paraffin.

To improve the solubility or the dispersibility of the active principle(s) in the waxy matrix, it is occasionally necessary to add an oil. This also makes it possible to adjust the melting point.

Besides the waxes mentioned above, the formulation according to the invention may contain an oil alone or as a mixture, chosen from hydrophobic silicone oils, cyclomethicones, lipophilic organofluorine oils, squalene and derivatives thereof, short-chain triglycerides and esters.

Other oily compounds such as oleyl alcohol, sunflower oil, palm oil, olive oil, fatty acids and fatty alcohols may be used, but the mixture obtained must be characterized by hydrophobic behaviour, absence of miscibility with water. A person skilled in the art knows that, for this waxy matrix, the heating temperature must not exceed the degradation temperature of at least one compound of the formulation.

Other compounds may be added to the waxy matrix. Mention may be made of fillers, such as talc, kaolin, colorants, and agents for adjusting the appearance, the colour, the density and the hardness of the matrix. For biological and pharmaceutical applications, a suitable composition, which is compatible in terms of toxicity, biocompatibility, absence of immunogenicity and biodegradability with oral absorption or topical application, should be chosen. The waxy matrix must be physiologically acceptable.

In certain embodiments of the invention, the waxy excipient(s) chosen from the group formed from fatty acids and fatty acid esters are in a mass proportion of between 0.5% and 85% by mass of the formulation, preferentially between 25% and 75%.

According to the invention, the formulation containing at least one active principle is endowed with properties of protecting the active principle(s). According to certain embodiments, the formulation according to the invention makes it possible to mask the taste, notably the taste of the active principle(s). According to certain embodiments, the formulation according to the invention makes it possible to modulate the release of the active principle(s).

According to one embodiment, the mixture may contain components such as pigments, metal oxides, iron and copper salts, and aluminium and silver salts. It may also contain biologically active compounds, essential oils, flavourings and other active substances. The filling capacity of the waxy matrix may extend from 0.2% to 85% relative to the weight. A person skilled in the art knows that when these components are incorporated into a waxy matrix according to the invention, a suitable composition of hydrophobic waxy excipients should be chosen so that the formulation according to the invention can be prepared.

Among the components that may be incorporated into the waxy matrix, mention may be made of carotenoids, free-radical scavengers, antiseptics, molecules acting on pigmentation or on inflammation, vitamins or provitamins A, B, C, D, E and PP and esters thereof.

The matrix contains an active principle or a mixture of active principles which may be in a dispersed or dissolved form or may be in both forms. Certain active agents may be totally or partially in amorphous form.

In certain embodiments, at least one active principle is chosen from phosphate derivatives, potassium salts, nutrients intended for human or animal nutrition, probiotic compounds, yeasts, biologically active compounds, flavourings, pharmaceutically active compounds, anticancer compounds, anti-inflammatory compounds, immunomodulatory compounds, immunosuppressant compounds, antibiotic compounds, hypolipaemic compounds, antithrombotic compounds, proton-pump-inhibiting compounds, compounds for veterinary use, vaccines, alkaloids, oligonucleotides, carotenoids, free-radical scavengers, hydroxy acids, molecules acting on pigmentation, vitamins and provitamins A, B, C, D, E and PP and esters thereof, pigments, carbon black, metal oxides, iron and copper salts, and aluminium and silver salts.

In this description, the term “pharmaceutical active principle” is used to denote any active therapeutic substance or mixture, which may advantageously be administered to humans or animals for diagnosing, caring for, reducing, treating or preventing the disease. By way of example, mention may be made of anticancer agents and anti-inflammatory agents, immunomodulators, immunosuppressants, antibiotics, hypolipaemic agents, antithrombotic agents, proton-pump inhibitors, vasodilators, vasoconstrictors, antidiuretic and diuretic agents, antiviral and antiretroviral agents, fibrates, antimalarial agents, veterinary compounds, vaccines, alkaloids, oligonucleotides, hormones, products for combating osteoporosis, octreotide, somatostatin and statins. In the present description, the term “nutrient” or “probiotic” or “food supplement” is used to denote any edible substance or mixture which may be advantageously administered to humans or animals for nutritional purposes. Examples that may be mentioned include vitamins, mineral salts, proteins, amino acids, edible trace elements, and microorganisms such as Saccharomyces boulardii used for their probiotic nature.

At least one active principle may be a biological catalyst. In the present description, the term “biological catalyst” is used to denote any molecule or microorganism or mixture which may be advantageously used in a fermentation or biotransformation process. An example that may be mentioned is Saccharomyces cerevisiae used in breadmaking processes. The incorporation of these yeasts into a hydrophobic waxy matrix according to the invention makes it possible to improve the quality of the dry forms in regions that are subject to harsh climatic conditions in hot and humid countries. The invention affords better stability at room temperature and longer conservation characterized by better fermenting power.

The formulation obtained in powder form according to the invention may be packaged in unit or multiple forms. In certain embodiments, the formulation according to the invention is a ready-to-use formulation. The formulation may be a conventional galenical form such as a gel capsule, a tablet, a wafer capsule, an orodispersible tablet, a lozenge obtained from freeze-drying, an aqueous suspension, a powder in a sachet, notably a dispersible powder in a sachet or a bottle, for obtaining a liquid form. Advantageously, a formulation according to the invention may be mixed with additives such as lubricants, for example talc, agents for improving the homogeneity such as silica, colorants, preserving agents, sweeteners and thickeners such as cellulose derivatives. The powder according to the invention may also be mixed with human or animal nutritional preparations.

In certain embodiments, the formulation according to the invention is contained in a unit or multiple container with a capacity of between 0.1 g and 1000 g.

The invention also relates to a process for preparing a formulation according to the invention.

In a preferential embodiment according to the invention, the powder is prepared according to a process comprising the following steps:
a/ preparation of the hydrophobic waxy matrix by melting said at least one waxy excipient with stirring at a temperature above the melting point of the mixture of said at least one waxy excipient. Advantageously, the waxy matrix is prepared a/by melting said at least one waxy excipient with stirring, followed by reducing the temperature to a temperature 3° C. above the melting point of the waxy matrix,
b/ addition and mechanical dispersion of the active principle(s) in the molten waxy matrix,
c/ recovery and solidification of the mixture of the waxy matrix and of said at least one active principle by cooling to at least 10° C. below the melting point of the mixture obtained. Advantageously, the mixture of the waxy matrix and of said at least one active principle is cooled to a temperature at least 10° C. below the melting point of said mixture, whereby the mixture solidifies,
e/ milling at a temperature at least 20° C. below the melting point of the matrix containing the active agent. Advantageously, mechanical milling of the solidified mixture is performed at a temperature at least 20° C. below the melting point of said mixture of the waxy matrix containing said at least one active principle,
f/ recovery of the powder comprising the active agent. The formulation according to the invention formed from a powder of solid particles with a size of between 5 μm and 3500 μm is obtained.

In certain embodiments, the process comprises a step d/ of premilling said solidified mixture prior to milling.

In a first step of the invention, said at least one active principle is dispersed in a wax or a mixture of waxes and of strictly hydrophobic excipients, named hydrophobic waxy matrix, which has been melted beforehand Said at least one active principle is protected against any contact with oxygen and water and more generally against any external chemical stress. The addition of said at least one active principle to the waxy matrix is performed above the melting point of the waxy matrix, at least 3° C. above and preferably 5° C. above the melting point of the waxy matrix, but always below the degradation or deactivation temperature of said at least one active principle. The liquid waxy matrix obtained, containing said at least one active principle, is then solidified by cooling and then optionally undergoes a coarse premilling step.

The matrix is then milled in order to obtain a ready-to-use powder of solid particles of controlled particle size. This particulate-matrix powder comprising said at least one active principle may be dispersed in water without any risk for the active agent, on account of its strictly hydrophobic nature.

This process is thus rapid to implement and does not necessitate any prior chemical modifications of the active agent or any surface treatment of the particles and/or of the crystals of active agent(s). It enables the active principle to be incorporated into the waxy matrix from the very first phase of mixing of the constituents of the waxy matrix. It is inexpensive and easy to perform.

The invention also relates to a formulation and a process for preparing such a formulation, characterized, in combination or separately, by all or some of the characteristics mentioned hereinabove or hereinbelow. Irrespective of the formal presentation that is given thereof, unless explicitly indicated otherwise, the various characteristics mentioned hereinabove or hereinbelow should not be considered as being strictly or inextricably linked together, the invention being able to concern only one of these structural or functional characteristics, or only a portion of these structural or functional characteristics, or only a portion of one of these structural or functional characteristics, or else any group, combination or juxtaposition of all or a portion of these structural or functional characteristics.

Other aims, characteristics and advantages of the invention will emerge on reading the following description, given without any implied limitation, of some of the possible embodiments thereof, and which refers to the attached figures in which:

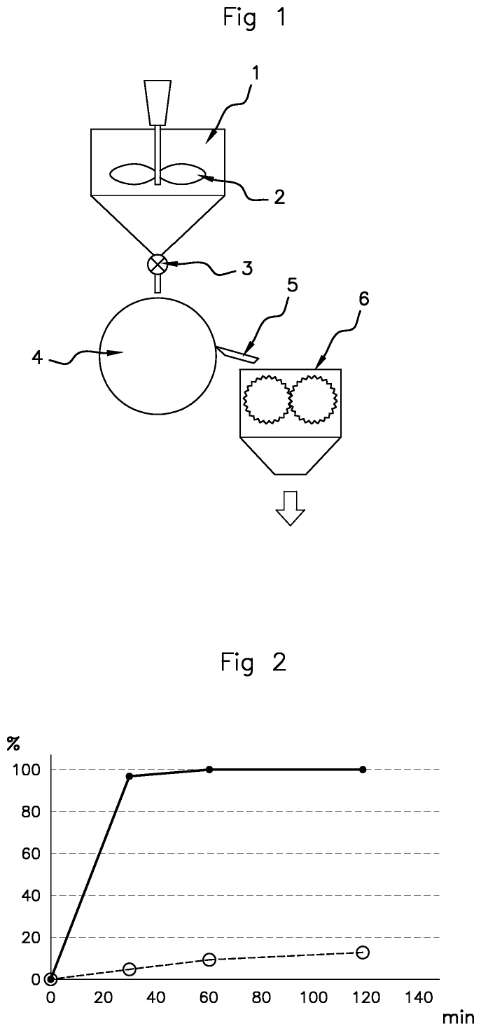

FIG. 1 is a schematic representation of a device for performing a process according to the invention, and

FIG. 2 is a graphic representation of a result obtained by a formulation according to the invention.

FIG. 2 is a graphic representation of the degradation in acidic medium (0.1 N HCl) of 2-([4-(3-methoxypropoxy)-3-methylpyridin-2-yl]methylsulfinyl)-1H-benzo[d]imidazole (BZ). FIG. 2 illustrates Example 1 and the improvement in the stability of the active agent (◯) formulated according to the invention relative to the non-formulated active agent (♦).

The process does not include any surfactant compound, any emulsifiers or any amphiphilic products in the formulation, nor does it require any organic solvents, the removal of which is always difficult and the use of which is increasingly restrictive. It does not involve any shear-thinning agent either. There is no contact of the formulation undergoing preparation with an organic or aqueous solvent or with water during the process. This prevents the dissolution and extraction of the water-soluble active agents that are capable of interacting with the active agents and of reducing the physicochemical stability, the storage time and the degree of filling. The active compound(s) or principle(s) are uniformly distributed in the divided waxy matrix, in contrast with other techniques.

The mixing of the various waxy components or excipients of the waxy matrix and of the active principle(s), constituting the composition from which the final formulation in powder form will be obtained, is performed in the first step of the process, in a thermostatically-maintained reactor, a melting vessel (1) or any other suitable system. The waxy excipient having the highest melting point is first melted, and the excipients are then successively added in order of decreasing melting point. In a preferential embodiment, the temperature is reduced, while always keeping it 3° C. above the melting point of the mixture obtained. The active agent is added last. It may prove necessary to modify the temperature as a function of the properties of the active agent, but always between 15° C. and 105° C.

A mechanical stirring method suitable for the homogeneous dispersion of all the components is applied. According to a preferred embodiment of the invention, the stirring device has the characteristic of having a paddle equipped with an impeller (2), preferentially a profiled three-blade impeller such as an Agimel TPE®, for dispersing the mixture of molten waxy excipients. The active agent, alone or as a mixture, is then added to the molten mixture. In a particular embodiment of the invention, the use of a T series Turrax® turbomixer or of an Agimel TTC® turbomixer may be necessary to obtain homogeneous dispersion of the active agent. The stirring time depends greatly on its nature. This mixing step is thus rapid and does not require a long and difficult mixing step.

There is nothing to prevent other suitable dispersion methods from being used, for example sonication, extrusion, static mixers or linear mixers, or recirculation pumps, while taking care, however, to ensure that the mixture obtained is indeed homogeneous.

The mixture obtained is immediately cooled so as to protect the most sensitive active principle(s) and to obtain a solid phase. According to a particular embodiment of the invention, for amounts of less than 1 kg, the cooling of the molten matrix containing the active principle(s) dispersed in the molten matrix may be performed by spreading on contact cooling plates. Solidification is obtained in less than 120 seconds, and the plates are then placed in a cold chamber, which enables the steps to be uncoupled, if necessary.

For larger amounts, cooling may be performed by passing through a continuous cooling system. An example that may be mentioned is the Sandvik model 321 stainless-steel continuous belt cooling system. According to a particular embodiment of the invention, the molten product is deposited in the form of droplets, filaments or a film onto a stainless-steel belt conveyor which passes through a cooling chamber and is recovered in solid form at the outlet.

Cooling is performed by heat exchange with a cold gas stream or by conduction with the cooled support. Depending on the amount of product and on the solidification temperature to be reached, a person skilled in the art will adjust the parameters of the system, in particular the flow rate of material, the throughput speed of the belt, the length and the temperature of the cooling chamber. In the case of certain equipment, the belt is also cooled, for example with liquid nitrogen.

In a particular embodiment, the reactor or melting vessel containing the mixture of the molten waxy matrix and of said at least one active principle is equipped with a pump for transferring at a controlled rate to the solidification system (3).

The cooling temperature of the mixture is regulated to at least 10° C. below the melting point of the mixture and preferentially to 15° C. below this temperature and, in a particular embodiment, to 45° C. below the melting point. According to one embodiment of the invention, the cooling temperature is between −195° C. and 45° C. and preferentially from −10° C. to 5° C.

In a particular embodiment of the invention, the melting vessel is equipped at the outlet with numerous dies (3) for producing molten matrix filaments. These filaments are continuously solidified by contact on a stainless-steel refrigerated rotating drum system (4) as described in FIG. 1. A scraper (5) positioned tangentially enables the solidified filaments to be detached and fragmented.

The fragmented mixture is then cooled by contact or convection before being tipped into the premilling system. It is necessary to premill the mixture comprising the solidified matrix, so as to obtain fragments preferentially with a size of less than 40 mm and particularly with a size of less than 5 mm, to be able to feed the mill correctly. To this end, use may be made of Retsch GM rotor, hammer or knife premills or jaw crusher premills such as the BB Series Jaw Crushers sold by the company Retsch, or cryogenic screw conveyors such as the WAM CX conveyors cooled by addition of liquid nitrogen or any other suitable system. Certain solidified mixtures are fragile and can be premilled and milled in the same machine in a single step, but at different speeds.

In a final step, the premilled matrix solidified by cooling is reduced to powder by milling (6). This final step makes it possible to obtain a formulation according to the invention in the form of a hydrophobic powder as final product. A large number of mills may be used, such as hammer, knife, rotor, ball or jet mills. Mention may be made of the APP® mill from the company Hosokawa Alpin AG, the FS®, L1A® and M5A® mills sold by the company Fitzpatrick or the SM, ZM and GM series mills from the company Retsch. To facilitate the milling of the waxy matrix, a preferential implementation of the invention consists in cooling the matrix to harden and embrittle it. This operation takes place before or during the milling step.

Cryomilling is a technique that is well known to those skilled in the art, as described in the patent FR2550961. Many milling systems can receive special equipment for cooling with liquid nitrogen or with cardice, such as the mill feed systems proposed by the company Fitzpatrick Co or Retsch. Certain feed systems may also be used for cooling the mixture. Examples that may be mentioned are WAM CX conveyors cooled by addition of liquid nitrogen. The particulate matrix powder obtained on conclusion of the milling can be packaged directly. The powder according to the invention has a particle size of between 5 μm and 3500 μm and preferentially between 10 μm and 2500 μm. In a preferential embodiment, the powder has a particle size of between 150 μm and 800 μm.

In a particular embodiment according to the invention, the molten mixture containing the active agent may be solidified and fragmented before milling via the cryopelletization technique described by Beteta and Ivanova in “Cool Down with Liquid Nitrogen”, CEP September 2015. By way of example, use may be made of the Cryogenic Pelletizer machine from the company CES. According to this technique, pellets of a few millimetres solidified from the molten matrix are obtained. This step is followed by the milling step.

The examples that follow are not limiting: they serve merely to illustrate the invention. For some of the following examples, the taste-masking tests were performed on a sample of 10 individuals. The test products are never absorbed.

The results are expressed according to the following scale:
1: the taste of the active principle is not detected,
2: the taste of the active principle is sparingly perceived,
3: the taste of the active principle is detected,
4: the taste of the active principle is still acceptable,
5: the taste of the active principle is unacceptable.

The test value is calculated by taking the mean of the grades obtained relative to the maximum grade out of 10.

Example 1: Preparation of a gastro-resistant formulation (BZ-AT) containing a benzimidazole derivative that is unstable in acidic medium The derivative referred to herein as (BZ) is 2-([4-(3-methoxypropoxy)-3-methylpyridin-2-yl]methylsulfinyl)-1H-benzo[d]imidazole.

Composition:

| Captex ® mixture of triglycerides (Abitec company) | 69.2 | g |
|---|---|---|
| DUB PP triglycerides (Stéarinerie Dubois) | 10 | g |
| BZ (Sigma): | 20 | g |
| Talc (Cooper) | 0.5 | |
| Sodium hydrogen carbonate (Cooper) | 0.2 | g |
| Silica (Aerosil) | 0.1 | g |

The compound with the highest melting point, the DUB PP triglycerides, is brought to 55° C. in a 500 ml thermostatically regulated reactor, and the various compounds are then gradually added from the highest melting point to the lowest. The temperature of the mixture is gradually lowered and then maintained at 45° C. During the addition of the composition, the stirring speed of the three-blade impeller is 100 rpm.

The BZ is added last. Dispersion of this active agent in the lipid phase is performed using a T25 Turrax brand turbomixer stirring system at a speed of 6000 rpm. The matrix is solidified by pouring onto stainless-steel plates whose temperature is maintained at −10° C. The recovered fragments, with a mean size of 35 mm, are premilled and then milled using a Retsch GM200 knife mill co-filled with cardice, under the following conditions:
Premilling: 20 seconds at 1500 rpm
Milling: 70 seconds at 3000 rpm

The particles thus obtained have a mean size of 342 μm.

Measurement of the stability of the particles in 0.1 N hydrochloric acid solution, via an HPLC assay method:
Column (ProntoSlL, Bischoff, Germany): C18, 5 μm, 120 Å, 4.6×150 mm,
Mobile phase: methanol, 65%/10 mM Na2HPO4, 35%,
Flow rate: 1.00 mL/min,
Temperature: 20° C. (thermostat),
Detection: UV (λ=310 nm)
Injection volume: 20 μL.

The stability is measured in 0.1 N HCl medium at 25° C., for a volume of 500 ml and 20 mg of BZ. The results collated in FIG. 2 are expressed as a percentage of the initial dose.

The BZ prepared in Example 1 (BZ-T5, ◯) shows degradation of less than 8% at 120 minutes of incubation. The degradation of non-formulated BZ (BZ, ♦) is 100%.

Test of gastroprotection and preliminary bioavailability on animals. In this test, the protective capacity of the BZ-AT formulation according to the invention as described in this example and of an unprotected form of BZ are compared.

Each formulation is administered orally to male Sprague-Dawley rats weighing between 175-250 g. Tests are performed with pure BZ and the BZ-AT form taken up at 10 mg/ml in the following aqueous solution:

| | | |
|---|---|---|
| Sucrose | 65 | g |
| Sodium methyl para-hydroxybenzoate | 0.015 | g |
| Sodium propyl para-hydroxybenzoate | 0.03 | g |
| Purified water | qs 100 | ml |

The volume that may be administered is adjusted as a function of each animal to enable a dose of 4 mL/kg to be achieved, i.e. 40 mg/kg of BZ. The administration is performed orally on the fasted animal. The results are collated in table 1 below.

TABLE 1

| | Analytical | | T max | C max | AUC |
|---|---|---|---|---|---|
| | score | Route | (h) | (ng/ml) | (ng · h/ml) |
| BZ-T5 | 0.987 | PO | 0.11 | 210.47 | 116.1 |
| BZ | 0.987 | PO | 0.8 | 2.54 | <5 |

Example 2: Preparation of powder containing taste-masked, gastro-protected, stabilized fish oil containing polyunsaturated fatty acids, EPA and DHA. Example given for the manufacture of 2.5 kg of particles containing fish oil with a high dose of Norwegian cod liver oil.

Composition:

| | | |
|---|---|---|
| Stearic acid | 0.61 | kg |
| PPM1 triglycerides (Stéarinerie Dubois) | 1.24 | kg |
| Fish oil (Solgar Norwegian Cod Liver Oil) | 0.625 | kg |
| Talc | 0.020 | kg |
| Silica | 0.005 | kg |

The compound with the highest melting point is brought 3° C. above its melting point in a 5 litre thermostatically regulated reactor, and the various compounds are then gradually added from the highest melting point to the lowest. The temperature of the mixture is gradually lowered and then maintained 3° C. above the melting point of the new mixture obtained, 48° C. in this instance. The fish oil is added last. Dispersion of these components in the molten waxy phase is performed using a stirring system equipped with an anchor-shaped spindle, at a speed of 200 rpm. Stirring is then performed using a T25 Turrax turbomixer at 4500 rpm for 6 minutes to obtain complete dispersion.

The matrix is solidified by pouring onto a rotating cylinder whose temperature is brought to 4° C.

The recovered fragments, with a mean size of 4 mm, are then cooled with cardice. The mixture is premilled and then milled using a stainless-steel Retsch GM knife mill
Speed: 3000 (rpm),
Time: 90 seconds,
Particle size: mean diameter of 445 μm.

This fish oil powder is then evaluated in a taste test. The result of the taste test gives a mean value of 1.20. The mean value is less than 2: the taste of the oil is undetectable.

Example 3: Preparation of flavoured powder containing taste-masked, gastro-protected, stabilized fish oil containing polyunsaturated fatty acids, EPA and DHA. This product is intended for the production of food supplements: Example given for the manufacture of 2.5 kg of mint-flavoured particles containing fish oil with a high dose of Norwegian cod liver oil.

Composition:

| | | |
|---|---|---|
| PPM1 triglycerides (Stéarinerie Dubois) | 1.24 | kg |
| Stearic acid | 0.608 | kg |
| Fish oil (Solgar Norwegian Cod Liver Oil) | 0.625 | kg |
| Talc | 0.020 | kg |
| Silica | 0.005 | kg |
| Mint flavouring | 0.002 | kg |

The powder is prepared according to the protocol described in Example 2. The powder obtained is characterized by the absence of detection of the fish oil taste, with a score of 1.2.

The powder obtained is characterized by a pronounced mint taste, with a score of 5 in the taste test.

Example 4: Preparation of an oral-route hydro-dispersible powder containing particles filled with BZ according to Example 1

Composition:

| | | |
|---|---|---|
| BZ-AT particles according to Example 1 | 100 | g |
| Mannitol | 80 | g |
| Flavouring | 10 | g |
| Aspartame | 8 | g |
| Xanthan gum (Xanthural 180) | 2 | g |

The composition is placed in a Turbula powder mixer (WAB France). After mixing, the powder is divided into 400 mg unit sachets.

Example 5: Preparation of particles containing stabilized ascorbic acid.

Composition:

| | | |
|---|---|---|
| Suppocire DM triglycerides (Stéarinerie Dubois) | 65 | g |
| Palmitic acid (Sigma) | 4 | g |
| Solid paraffin (Sigma) | 1 | g |
| Ascorbic acid (Sigma) | 30 | g |

The compound with the highest melting point is brought 3° C. above its melting point in a thermostatically regulated container, and the various compounds are then gradually added from the highest melting point to the lowest. During the addition of the compounds, the stirring speed of the three-blade impeller is 180±20 rpm. Stirring is maintained for 60 seconds after the end of the addition. The temperature of the mixture is gradually lowered and then maintained 5° C. above the melting point of the new mixture obtained. The ascorbic acid is added last. Dispersion of this component is performed using a T25 Turrax brand turbomixer stirring system at a speed of 3000 rpm for 3 minutes.

The matrix is solidified by pouring onto stainless-steel plates whose temperature is maintained at −4° C. The recovered fragments, with a mean size of less than 15 mm, are premilled and then milled using an IKA M20 mill thermostatically maintained at −10° C., under the following conditions:
premilling: 30 seconds at 1500 rpm,
milling: 90 seconds at 6000 rpm.

The final particle size measured is characterized by a mean particle diameter of 345 μm.

Example 6: Preparation of a gastro-protected, stabilized live-yeast powder according to the procedure of Example 2:

| | | |
|---|---|---|
| DUB triglycerides (Stéarinerie Dubois) | 650 | g |
| Suppocire CM triglycerides | 100 | g |
| Stearic acid | 50 | g |
| Desiccated Saccharomyces cerevisiae | 720 | g |
| Hydrophobic silica | 5 | g |

The powder obtained has the following characteristics:
particle size=910 μm,
concentration of viable cells:
   initial powder: 2.8×10¹⁰ CFU/g,
   matrix powder containing the yeasts: 1.72×10¹⁰ CFU/g.

The invention may be the subject of numerous variants and applications other than those described hereinabove. In particular, it goes without saying that, unless otherwise indicated, the various structural and functional characteristics of each of the embodiments described hereinabove must not be considered as combined and/or strictly and/or inextricably linked to each other, but, on the contrary, as simple juxtapositions. In addition, the structural and/or functional characteristics of the various embodiments described hereinabove may form the subject totally or partly of any different juxtaposition or of any different combination.

1/—Hydrophobic gastro-protected formulation of at least one active principle, characterized in that it is in the form of solid particles with a size of between 5 μm and 3500 μm, the solid particles of the formulation comprising:
at least one waxy excipient forming a waxy matrix; and
said at least one active principle chosen from the group formed from active principles for agrifood use, active principles for veterinary use, active principles for probiotic use and active principles for pharmaceutical use;
the solid particles being strictly hydrophobic, non-hygroscopic, non-injectable, free of water, free of surfactants, free of emulsifiers, free of solvent, and free of shear-thinning polymer;
the solid particles having a melting point of between 15° C. and 60° C.;
said at least one active principle being uniformly distributed in the waxy matrix in a mass proportion of between 0.2% and 85%;
said solid particles being suitable for protecting and stabilizing said at least one active principle in a gastric medium and being dispersible in an intestinal medium.

2/—Formulation according to claim 1, wherein at least one waxy excipient of the waxy matrix is chosen from the group formed from triglycerides and derivatives thereof, palm oil, carnauba wax, candelilla wax, esparto grass wax, cocoa butter, plant waxes, olive wax, rice wax, hydrogenated jojoba wax, absolute waxes of flowers, beeswaxes, modified beeswaxes, polyolefins, non-neutralized and non-ionized fatty acids, esters of linear-chain fatty acids with a number of carbon atoms of between 4 and 30, for instance lauric acid, myristic acid, palmitic acid and stearic acid, esters of linear-chain fatty acids with a number of carbon atoms of between 4 and 30 and hydrophobic lipids.

3/—Formulation according to claim 1, wherein the waxy excipients chosen from the group formed from fatty acids and fatty acid esters are in a mass proportion of between 0.5% and 85% of the mass of the formulation, preferentially between 25% and 75%.

4/—Formulation according to claim 1, wherein said formulation is in the form of hydrophobic solid particles with a size of between 10 μm and 2500 μm and preferentially between 150 μm and 800 μm.

5/—Formulation according to claim 1, wherein the melting point is between 25° C. and 60° C., preferentially between 32° C. and 52° C.

6/—Formulation according to claim 1, wherein at least one active principle is chosen from phosphate derivatives, potassium salts, nutrients intended for human or animal nutrition, probiotic compounds, yeasts, biologically active compounds, flavourings, pharmaceutically active compounds, anticancer compounds, anti-inflammatory compounds, immunomodulatory compounds, immunosuppressant compounds, antibiotic compounds, hypolipaemic compounds, antithrombotic compounds, proton-pump-inhibiting compounds, compounds for veterinary use, vaccines, alkaloids, oligonucleotides, carotenoids, free-radical scavengers, hydroxy acids, molecules acting on pigmentation, vitamins and provitamins A, B, C, D, E and PP and esters thereof, pigments, carbon black, metal oxides, iron and copper salts, and aluminium and silver salts.

7/—Formulation according to claim 1, wherein said formulation is ready to use in the form of a powder in a sachet, a tablet, a lozenge obtained by freeze-drying, a gel capsule, a wafer capsule, or a powder for reconstituting a liquid composition in a bottle by adding water.

8/—Process for preparing a formulation according to claim 1, wherein said process comprises the following steps:
a/ preparation of the waxy matrix by melting said at least one waxy excipient with stirring, followed by reducing the temperature to a temperature 3° C. above the melting point of the waxy matrix,
b/ addition and dispersion of said at least one active principle in the molten waxy matrix,
c/ cooling of the mixture of the waxy matrix and of said at least one active principle to a temperature at least 10° C. below the melting point of said mixture, whereby the mixture solidifies,
e/ mechanical milling of the solidified mixture at a temperature at least 20° C. below the melting point of the waxy matrix containing said at least one active principle,
f/ whereby a powder of solid particles with a size of between 5 μm and 3500 μm is formed.

9/—Process according to claim 8, wherein step e/ of milling the waxy matrix containing said at least one active principle is performed using a hammer mill or a knife mill or a disc mill or a jaw crusher or a ball mill or a jet mill.

10/—Process according to claim 8, wherein the solidification, premilling and/or milling steps are performed by cooling with cardice or with liquid nitrogen.

11/—Process according to claim 8, wherein step c/ of cooling the waxy matrix is performed by pouring the waxy matrix on a refrigerated rotating drum comprising a tangential knife for detaching the solidified waxy matrix and concomitantly premilling it.

12/—Process according to claim 8, wherein step e/ is performed using a mill fed via a screw conveyor refrigerated by addition of liquid nitrogen or cardice, enabling the premilling and the feeding of the mill in a single step.
